(19) United States
(12) Patent Application Publication
(10) Pub. No.: US 2020/0269033 A1
(43) Pub. Date: Aug. 27, 2020

(54) DISINFECTING CAP FOR LUER DEVICES

(71) Applicant: 3M INNOVATIVE PROPERTIES COMPANY, SAINT PAUL, MN (US)

(72) Inventors: THOMAS R. J. CORRIGAN, SAINT PAUL, MN (US); RYAN D. ERICKSON, ROSEVILLE, MN (US); ALAN R. DOMBROWSKI, WOODBURY, MN (US)

(73) Assignee: 3M INNOVATIVE PROPERTIES COMPANY, SAINT PAUL, MN (US)

(21) Appl. No.: 16/761,572

(22) PCT Filed: Nov. 2, 2018
(86) PCT No.: PCT/IB2018/058641

§ 371 Date: May 5, 2020

Related U.S. Application Data
(60) Provisional application No. 62/583,534, filed on Nov. 9, 2017.

(51) Int. Cl. A61M 39/16; A61M 39/20

(52) U.S. Cl. CPC A61M 39/162; A61M 2205/0205; A61M 2207/00; A61M 39/20

(57) ABSTRACT

An article comprises a cap and a cover. The cap comprises an opening to an interior cavity, and has an inner surface defining the interior cavity and an outer surface. The cover is disposed within the interior cavity of the cap near the opening. The cover comprises a base having an inner surface facing the cap opening and an opposite outer surface. A sidewall of the cover extends from a perimeter of the base toward the cap opening. The sidewall has an outer surface facing the inner surface of the cap and an opposite inner surface. Micro features are provided on at least the inner surface of the sidewall. The cover is displaceable into the interior cavity of the cap.

### TECHNICAL FIELD

This application relates to disinfecting caps for luer devices and methods of making such caps.

### BACKGROUND

A luer is a standardized system of fluid fittings, ports, and interfaces used for making fluid-tight connections between medical implements. For instance, some male luers include a tapered male protrusion defining a lumen, where the protrusion extends out from a sleeve or chamber that has internal threads on an inner wall of the chamber. A luer lock or other female port with or without an external thread can be fitted into the sleeve and over the male protrusion, for a friction-based fitting on the male protrusion. A male luer can be used on syringes, injection ports, or other intravenous (IV) lines.

### SUMMARY

Some embodiments are directed to an article comprising a cap and a cover. The cap comprises an opening to an interior cavity, and has an inner surface defining the interior cavity and an outer surface. The cover is disposed within the interior cavity of the cap near the opening. The cover comprises a base having an inner surface facing the cap opening and an opposite outer surface. A sidewall of the cover extends from a perimeter of the base toward the cap opening. The sidewall has an outer surface facing the inner surface of the cap and an opposite inner surface. Micro features are provided on at least the inner surface of the sidewall. The cover is displaceable into the interior cavity of the cap.

Other embodiments are directed to a disinfectant system comprising a cap and a cover. The cap comprises an opening to an interior cavity, and has an inner surface defining the interior cavity and an outer surface. The cover is disposed within the interior cavity of the cap near the opening. The cover comprises a base having an inner surface facing the cap opening and an opposite outer surface. A sidewall of the cover extends from a perimeter of the base toward the cap opening. The sidewall has an outer surface facing the inner surface of the cap and an opposite inner surface. Micro features are provided on the inner surfaces of the base and the sidewall. Disinfectant is provided in the interior cavity of the cap. The cover is displaceable into the interior cavity of the cap.

Further embodiments are directed to method of making an article comprising a cap and a cover. The method comprises providing a foldable cover that comprises a base and a sidewall of the cover in a single plane. The method comprises placing the foldable cover over an opening of the cap such that the base is centered on the opening. The method also comprises pushing the foldable cover through the cap opening, the sidewall bending at a perimeter of the base as the foldable cover progresses into an interior cavity of the cap.

These and other aspects of the present application will be apparent from the detailed description below. In no event, however, should the above summaries be construed as limitations on the claimed subject matter, which subject matter is defined solely by the attached claims.

### BRIEF DESCRIPTION OF THE DRAWINGS

FIG. 1 is a cross-sectional view of a disinfectant system comprising a cap and a foldable cover in accordance with various embodiments;

FIG. 2 is an exploded view of the disinfectant system shown in FIG. 1, with the foldable cover shown spaced apart from the cap;

FIG. 3 shows a disinfectant system comprising a cap and a foldable cover secured to a luer device;

FIG. 4 is a cross-sectional perspective view showing aspects of a foldable cover installed in an interior cavity of the cap in accordance with various embodiments;

FIG. 5 illustrates a foldable cover in a flat state in accordance with various embodiments;

FIG. 6 shows the foldable cover of FIG. 5 in a folded state;

FIG. 7 illustrates a foldable cover in a flat state in accordance with various embodiments;

FIG. 8 shows the foldable cover of FIG. 7 in an initial flat state and a final folded state;

FIG. 9 illustrates a groove defining a hinge point between a base and each of a multiplicity of segments of a foldable cover in accordance with various embodiments;

FIGS. 10 and 11 show a disinfectant system comprising a cap and a cover comprising a perforated basket structure in accordance with various embodiments;

FIG. 12 shows a foldable cover of a disinfectant system comprising micro features in accordance with various embodiments;

FIG. 13 shows a foldable cover of a disinfectant system comprising micro features in accordance with some embodiments;

FIG. 14 shows a foldable cover of a disinfectant system comprising micro features in accordance with other embodiments;

FIG. 15 shows a foldable cover of a disinfectant system comprising micro features in accordance with further embodiments;

FIG. 16 shows a foldable cover of a disinfectant system comprising micro features in accordance with various embodiments;

FIG. 17 shows a foldable cover of a disinfectant system comprising micro features in accordance with some embodiments;

FIG. 18 shows a foldable cover of a disinfectant system comprising micro features in accordance with other embodiments;

FIG. 19 shows a foldable cover of a disinfectant system comprising a substrate and a layer of porous fiber-like material affixed to the substrate in accordance with further embodiments;

FIG. 19A shows a close-up of the layer of porous fiber-like material in FIG. 19;

FIG. 20 shows a foldable cover of a disinfectant system comprising a substrate and a layer of sponge material affixed to the substrate in accordance with various embodiments;

FIG. 20A shows a close-up of the layer of sponge material in FIG. 20;

FIG. 21 illustrates a foldable cover of a disinfectant system in a flat state positioned relative to an insertion tool prior to the foldable cover being inserted into a cap of the disinfecting system in accordance with various embodiments;

FIG. 22A shows retention features of the foldable cover and the insertion tool shown in FIG. 21 in accordance with various embodiments;

FIG. 22B shows the flat foldable cover of FIG. 21 being retained against a shaft of the insertion tool;

FIG. 23 shows the flat foldable cover retained against the shaft of the insertion tool prior to the foldable cover being inserted into an interior cavity of the cap;

FIG. 24 shows the segments of the foldable cover being bent as the foldable cover and shaft of the insertion tool are advanced into the interior cavity of the cap;

FIG. 25 shows the foldable cover inserted to its predetermined insertion depth within the interior cavity of the cap; and

FIG. 26 shows a disinfecting system comprising the foldable cover installed within the interior cavity of the cap.

### DETAILED DESCRIPTION

Embodiments of the present disclosure are directed to disinfecting caps for use with luer devices, such as a male luer end of an IV catheter set. A luer is a standardized system of fluid fittings, ports, and interfaces that can be used to make fluidic connections between medical implements. A disinfecting cap of the present disclosure is configured to disinfect surfaces of a luer device protected by the cap.

Embodiments of the disclosure address the need for a low-cost device that can protect and disinfect a luer device while reducing or eliminating the risk of introducing a liquid disinfectant into a lumen of the luer device. Exemplary embodiments provided below are directed to a two-part disinfectant system comprising a cap and a foldable cover within the cap. The cap and foldable cover cooperate to actively disinfect surfaces of a luer device when the cap is secured to the luer device.

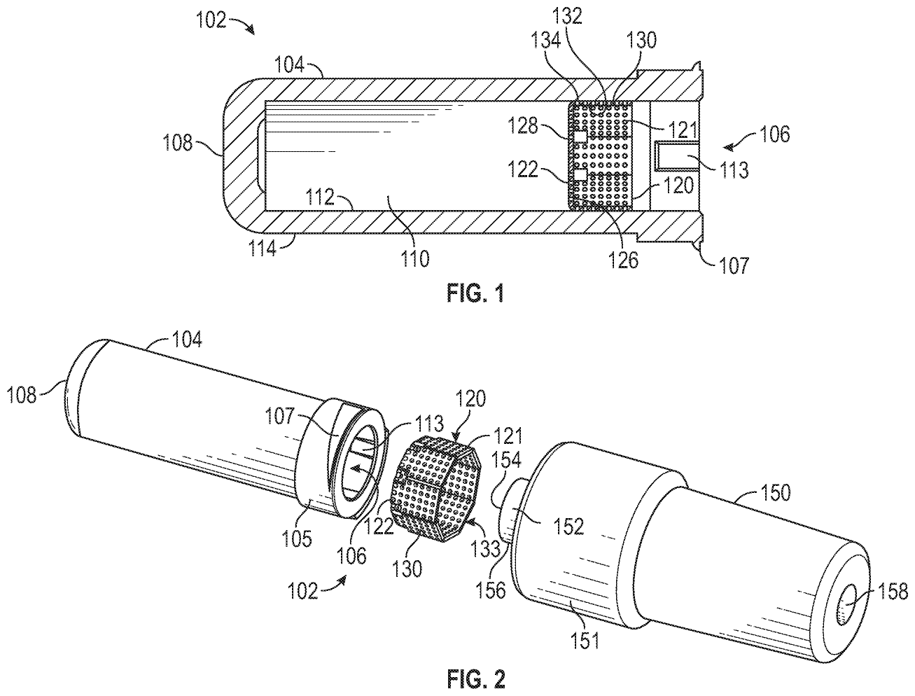

FIG. 1 is a cross-sectional view of a disinfectant system 102 in accordance with various embodiments. In FIG. 1, the disinfectant system 102 is shown in a pre-activation state. The disinfectant system 102 comprises a cap 104 and a cover 120. The cap 104 includes a closed end 108 and an opening 106 to an interior cavity 110. The cap 104 also includes an inner surface 112 defining the interior cavity 110 and an outer surface 114. Although not shown in FIG. 1, the outer surface 114 of the cap 104 can include a number of gripping features spaced around the periphery of the outer surface 114. The gripping features facilitate manual manipulation of the cap 104 during use.

The cover 120 is disposed within the interior cavity 110 of the cap 104 near the opening 106. As will be described in detail hereinbelow, the cover 120 is displaceable into the interior cavity 110 of the cap 104. The cover 120 includes a base 122 having an inner surface 126 facing the cap opening 106 and an opposite outer surface 128. The cover 120 also includes a sidewall 130 extending from a perimeter of the base 122 toward the cap opening 106. The sidewall 130 includes an outer surface 134 facing the inner surface 112 of the cap 104 and an opposite inner surface 132. The cover 120 remains substantially stationary in its pre-activation state due to an interference fit within the interior cavity 110 of the cap 104. In some embodiments, the sidewall 130 of the cover 120 exerts a spring-like force against the inner surface 112 of the cap 104. A stop feature 113 is situated near the opening 106 to prevent the cover 120 from exiting the interior cavity 110 of the cap 104. The stop feature 113 can be a bump, step or ledge feature, for example. One or more of the stop features 113 can be situated along a perimeter of the inner surface 112 near the opening 106.

The cover 120 includes micro features 121 on the inner surface 132 of the sidewall 130 and, in some embodiments, on the inner surface 126 of the base 122. The micro features 121 can include through-holes, slots, apertures and/or perforations that extend all the way through the sidewall 130. Alternatively, or in addition, the micro features 121 can include blind-holes, open channels and/or depressions formed on the inner surface 132 of the sidewall 130 that do not extend all the way through the side wall 130. In some embodiments, the sidewall 130 comprises more than one type of micro feature 121 (e.g., a combination of apertures and channels). In some embodiments, the micro features 121 are formed in both the sidewall 130 and base 122 of the cover 120.

A cover of the present disclosure, such as cover 120, can be molded from various materials that include, but is not limited to, composite materials, polymeric materials (e.g., elastomeric, thermoplastic, thermoset, biodegradable, or combinations thereof), or combinations thereof. Examples of elastomeric materials can include silicones, polydimethylsiloxane (PDMS), liquid silicone rubber, poly(styrene-butadiene-styrene), other suitable thermoplastic elastomers, and combinations thereof. Examples of thermoplastic materials can include one or more of polyolefins (e.g., polyethylene (high density polyethylene (HDPE), medium density polyethylene (MDPE), low density polyethylene (LDPE), linear low density polyethylene (LLDPE), metallocene polyethylene, and the like, and combinations thereof), polypropylene (e.g., atactic and syndiotactic polypropylene)), polyamides (e.g. nylon), polyurethane, polyacetal (such as Delrin), polyacrylates, and polyesters (such as polyethylene terephthalate (PET), polyethylene terephthalate glycol (PETG), and aliphatic polyesters such as polylactic acid), fluoroplastics (such as THV from 3M company, St. Paul, Minn.), and combinations thereof. Examples of thermoset materials can include one or more of polyurethanes, silicones, epoxies, melamine, phenol-formaldehyde resin, and combinations thereof. Examples of biodegradable polymers can include one or more of polylactic acid (PLA), polyglycolic acid (PGA), poly(caprolactone), copolymers of lactide and glycolide, poly(ethylene succinate), polyhydroxybutyrate, and combinations thereof.

A cover of the present disclosure can also be made from a metal material such as copper, aluminum, etc., or a combination of metal and plastic. A cover can also be made from a non-woven, fibrous or porous material such as papers, foams, porous plastics, or spun bond, melt blown or electrospun materials.

According to various embodiments, the micro features 121 of the cover 120 are configured to retain a liquid disinfectant or cleaning agent. The liquid disinfectant or cleaning agent can be any substance or material that cleans a surface of bacterial and/or viral microorganisms and includes alcohols (e.g., isopropyl alcohol and ethanol), alcohols at various concentrations (e.g. 70%/30% V/V isopropyl alcohol/water), chlorhexidine (chlorhexidine gluconate, chlorhexidine acetate), povidone-iodine, hydrogen peroxide, soap, hydrochloric acid, chloroxylenol (PMCX), PHMB (polyhexamethylene biguanide), octenidene, benzalkonium chloride, and combinations thereof.

The term “micro feature” as used herein generally refers to a feature that can 1) retain liquid via surface wettability enhancement, 2) retain liquid via volumetric holding capacity, and/or 3) transport liquid via capillary action. The micro features are shaped and sized differently according to which function they are meant to perform. Additionally, due to the underlying physics, the micro features can be sized to the combination of working fluid (e.g., a disinfectant) and solid material of the cover being utilized in the application.

For example, Equation 1 below can be used to design a rectangular channel that transports liquid by capillary action. The liquid and solid material used to make up the cap are chosen to produce a net positive capillary pressure. In practice, this can be achieved by selecting a solid material with a critical surface tension which is close to or higher than the liquid's surface tension. This, as a first step, would ensure good surface wettability with low contact angles (e.g., 0≤θ<90°). The pumping capacity of such a channel is then dictated by the liquid surface tension and channel dimensions.

$${P}_{c} = γ ( \frac{cos {θ}_{b} + cos {θ}_{t}}{h} + \frac{cos {θ}_{r} + cos {θ}_{l}}{w} ) \qquad Equation [ 1 ]$$

wherein, P_{c}=capillary pressure, γ=liquid surface tension, θ_{b}=liquid contact angle at bottom of channel, θₜ=liquid contact angle at top of channel, θᵣ=liquid contact angle at right side of channel, θₗ=liquid contact angle at left side of channel, h=channel height, and w=channel width.

The illustrative example above highlights the need for linking micro feature size to its specific functionality. In general, for the cases where liquid transport is desired, channels typically have cross-sectional dimensions which are smaller than 0.02 inches (0.5 mm). This dimension can be decreased when capillarity must be increased to compensate for gravitational forces. For the same behavior, a nonwoven surface may be chosen with fiber diameters of <0.004 inches (100 μm) to encourage capillarity. Should liquid retention be desired, dimensions are only limited by the requirement to fit within the luer cover. Volumetric capacity of the feature, not a particular length scale, is most important in this situation.

The disinfectant system 102 shown in FIG. 1 is configured for use with a male luer device 150, such as that shown in FIG. 2. FIG. 2 is an exploded view of the disinfectant system 102, with the cover 120 shown spaced apart from the cap 104 for purposes of illustration. The cap 104 comprises a flange 105 which includes a thread 107. The luer device 150 includes a collar 151 comprising a thread (not shown in FIG. 2). The cap 104 can be secured to the luer device 150 by advancing the flange 105 of the cap 104 over a male-taper fitting 152 and rotating the cap 104 relative to the luer device 150 in a first direction (e.g., clockwise) to engage the threads. The cap 104 can be removed from the luer device 150 by rotating the cap 104 relative to the luer device 150 in the opposite direction (e.g., counterclockwise). In some embodiments, the cap 104 and luer device 150 are devoid of a thread, and can be secured to one another, for example, via a press-fit connection.

The male-taper fitting 152 is dimensioned to be received by the opening 106 and interior cavity 110 of the cap 104. The male-taper fitting 152 has a lumen 158 and a taper that corresponds to a taper of the inner surface 112 of the cap 104. According to various embodiments, the taper of the male-taper fitting 152 preferably conforms to a known industry-standard, such as ISO 594-2 (e.g., a 6% luer taper).

When the cap 104 is advanced over the male-taper fitting 152, the male-taper fitting 152 comes into contact with the cover 120 positioned near the opening 106 of the cap 104. The sidewall 130 of the cover 120 includes an opening 133 dimensioned to receive the male-taper fitting 152. In an activation state of the disinfectant system 102, contact between the male-taper fitting 152 and the cover 120 results in the transfer of liquid disinfectant from the cover 120 to the male-taper fitting 152. A first phase of the activation state involves disinfecting of the male-taper fitting 152 while the cover 120 remains substantially stationary within the cap 104. A second phase of the activation state involves displacement of the cover 120 into the inner cavity 110 of the cap 104 as the cap 104 is secured to the luer device 150.

During the first phase of the activation state of the disinfecting system 102, a side surface 156 of the male-taper fitting 152 contacts the inner surface 132 of the sidewall 130 as the male-taper fitting 152 is advanced toward the base 122 of the cover 120. The cover 120 remains substantially stationary while the male-taper fitting 152 contacts the inner surface 132 of the sidewall 130. More particularly, the cover 120 remains substantially stationary until a face 154 of the male-taper fitting 152 contacts the inner surface 126 of the base 122. The liquid disinfectant retained by the micro features 121 is transferred to the side surface 156 of the male-taper fitting 152 due to intimate contact between the side surface 156 and the inner surface 132 of the sidewall 130. Rotation of the male-taper fitting 152 when in contact with the inner surface 132 of the sidewall 130 can also facilitate disbursement of the liquid disinfectant over the side surface 156 of the male-taper fitting 152.

When the face 154 of the male-taper fitting 152 contacts the base 122 of the cover 120, liquid disinfectant retained by the micro features 121 of the inner surface 126 of the base 122 is transferred to the face 154 of the male-taper fitting 152. Rotation of the male-taper fitting 152 when in contact with the inner surface 126 of the base 122 can facilitate disbursement of the liquid disinfectant over the face 154 of the male-taper fitting 152.

During the second phase of the activation state of the disinfecting system 102, the distal end of the male-taper fitting 152 is encompassed by the cover 120. In this configuration, the face 154 of the male-taper fitting 152 is in contact with the inner surface 126 of the base 122 of the cover 120, and the side surface 156 of the male-taper fitting 152 is in contact with the inner surface 132 of the sidewall 130 of the cover 120. Forcible attachment of the cap 104 to the luer device 150 causes the male-taper fitting 152 to move the cover 120 into the inner cavity 110 toward the closed end 108 of the cap 104.

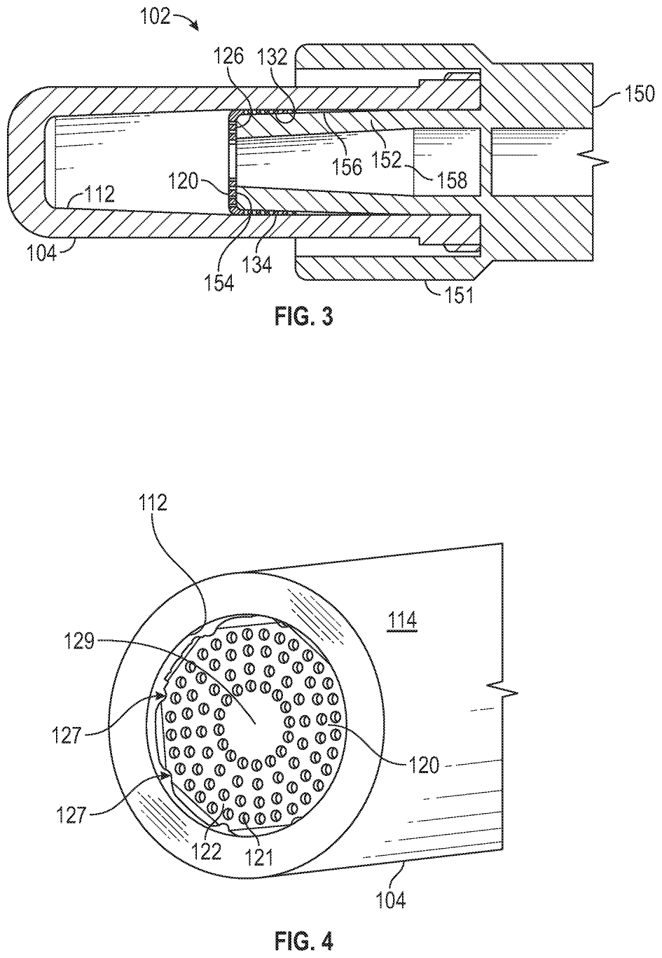

After the cap 104 is secured to the luer device 150, the disinfecting system 102 is in a post-activation state, which is shown in FIG. 3. In the post-activation state, the cap 104 remains securely attached to the luer device 150, thereby maintaining sterility of the male-taper fitting 152. As is best seen in FIG. 3, a clearance is added between the inner surface 112 of the cap 104 and the side surface 156 of the male-taper fitting 152 to accommodate the thickness of the sidewall 130 of the cover 120. In some embodiments, such as that shown in FIG. 3, the inner surface 112 of the cap 104 can be tapered. The taper of the inner surface 112 can be chosen to change the bending location of the sidewall 130 relative to the base 122 to accommodate the changing diameter in the tapered portion.

To detach the cap 104 from the lure device 150, the cap 104 is rotated in the appropriate direction (e.g., counterclockwise) then pulled away from the lure device 150. In some embodiments, the cover 120 remains in contact with the distal end of the male-taper fitting 152 as the cap 104 is detached from the luer device 150. As such, the cover 120 is displaced from its post-activation state shown in FIG. 3 and is moved toward the opening 106 of the cap 104. As is shown in FIGS. 1 and 2, one or more stop features 113 near the opening 106 of the cap 120 prevent the cover 120 from exiting the interior cavity 110 of the cap 104. The cover 120 is stripped from the male-taper fitting 152 when the cover 120 contacts the stop features 113 while the male-taper fitting 152 is removed from the interior cavity 110 of the cap 104.

According to some embodiments, the sole source of the liquid disinfectant transferred to the male-taper fitting 152 is the micro features 121 on the cover 120. As such, the interior cavity 110 of the cap 104 is devoid of a reservoir of liquid disinfectant. Retaining the liquid disinfectant within the micro features 121 on the cover 120 significantly reduces or eliminates the risk of introducing liquid disinfectant into the lumen 158 of the lure device 150.

In accordance with other embodiments, a reservoir of liquid disinfectant is provided in the interior cavity 110 of the cap 104. In embodiments that utilize a reservoir of liquid disinfectant, and as shown in FIG. 4, the cover 120 includes a number of notches 127 distributed around the perimeter of the base 122. The notches 127 define gaps between the base 122 of the cover 120 and the inner surface 112 of the cap 104. When the cap 140 is forcibly attached to the male lure 150, the male-taper fitting 152 forces the cover 120 into the reservoir of liquid disinfectant, causing the disinfectant to flow through the notches 127. The disinfectant that flows through the notches 127 saturates the micro features 121 on the inner surface 132 of the sidewall 130. The disinfectant received by the micro features 121 via the notches 127 is wicked to the side surface 156 of the male-taper fitting 152. The disinfectant is also transferred to the face 154 of the male-taper fitting 152 via the micro features 121 on the base 122 of the cover 120. In the embodiment shown in FIG. 4, the base 122 includes a solid center region 129 that inhibits disinfectant from passing into the lumen 158 of the male-taper fitting 152. The solid region 129 preferably has a diameter slightly larger than that of the opening of the lumen 158 on the face 154 of the male-taper fitting 152.

According to various embodiments, the cover of a disinfecting cap, such as that shown in FIGS. 1-4, is fabricated from flat sheet material, such as plastic material, metal material, or porous fiber-like material. The flat sheet material can be processed to incorporate various types of micro features, such as through-holes, slots, blind-holes, open channels, perforations, apertures, or a combination of these features. In some embodiments, a flat substrate, such as a plastic or metal substrate, supports a layer of material comprising micro features (e.g., a porous fiber-like material, nonwoven material or sponge material). The layer of material comprising micro features can be adhesively attached or otherwise bonded to the flat substrate.

The flat sheet material or substrate is formed to include a circular base and a multiplicity of segments projecting from and spaced along the perimeter of the base. The segments can be folded relative to the base to form a sidewall of the cover. Typically, the segments are folded at an angle of about 90° relative to the base to define a folded insert which is positioned within the interior cavity of a disinfecting cap, as is shown in FIG. 1. In some embodiments, the segments are bent to form the folded cover during fabrication of the cover, such as during a stamping process. In such embodiments, the pre-bent cover is subsequently inserted into the interior cavity of a disinfecting cap. According to other embodiments, the segments of the cover are automatically folded relative to the base during a process of inserting the cover into the interior cavity of the disinfecting cap (see, e.g., FIGS. 21-26).

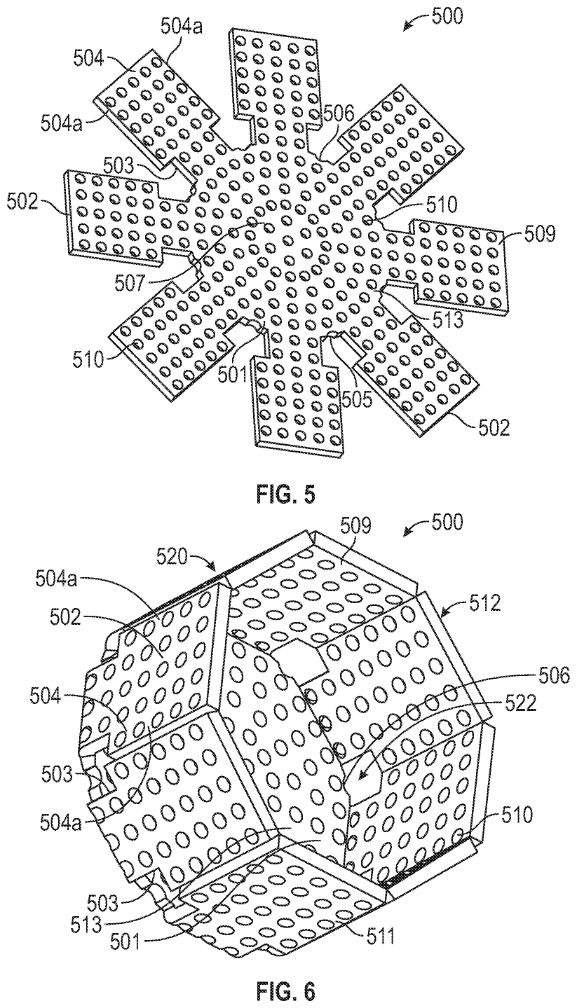

FIG. 5 illustrates a foldable cover 500 in accordance with various embodiments. The cover 500 in FIG. 5 is shown in a flat state. FIG. 6 shows the cover 500 of FIG. 5 in a folded state. For illustrative purposes, the cover 500 in FIG. 5 is shown to include a first type of micro features (blind-holes), and the cover in FIG. 6 is shown to include micro features of a second type (through-holes). As is best seen in FIG. 5, the cover 500 includes a base 501 having a generally circular shape. The base 501 has a diameter that is about the same as the diameter of the inner cavity of the disinfecting cap, and allows for an interference fit between the cover 500 and the inner surface of the disinfectant cap.

The base 501 has a perimeter 505 from which a multiplicity of segments 502 project. The segments 502 are spaced apart from one another and distributed along the perimeter 505 of the base 501. For example, the segments 502 can be symmetrically arranged around the perimeter 505 of the base 501 and equidistant from each other. In the embodiment shown in FIG. 5, the cover 500 includes eight segments 502. It is understood that the cover 500 can include more or fewer than eight segments 502. Each of the segments 502 includes a narrow region 503 adjacent the base 501 and a wide region 504 adjacent the narrow region 503. The wide region 504 defines “wings” 504a of the segments 502. In the embodiment shown in FIG. 5, the perimeter 505 of the base 501 includes a number of notches 506. A notch 506 is situated on the perimeter 505 between adjacent segments 502. In some embodiments, the base 501 is devoid of notches 506.

FIG. 6 shows the cover 500 of FIG. 5 in a folded state. As shown, the segments 502 are folded relative to the base 501 at an angle of about 90°. In the folded state, the wings 504a of adjacent wide regions 504 contact one another to define a continuous sidewall 520 of the cover 500. The folded cover 500 has an opening 512 dimensioned to receive a distal end of a male-taper fitting of a luer device. As can be seen in FIG. 6, a gap 522 is formed between the narrow regions 503 of adjacent segments 502 when the segments 502 are in their folded state. The gap 522 defines an opening on the sidewall 520 of the cover 500. The gaps 522 create a weaker cross section to help control the bending of the wings 504. The gaps 522 can also comprise the same opening as the notch 506 and can allow liquid to flow from the reservoir to the side surface of the male-taper fitting.

In some embodiments, the inner surface 509 of the segments 502 incorporate micro features 510. In other embodiments, the inner surface 509 of the segments 502 and an inner surface 513 of the base 501 incorporate micro features 510. As is shown in FIG. 5, the micro features 510 can include blind-holes that do not extend through the base 501 and segment 502 of the cover 500. As such, the outer surfaces of the cover 500 can be devoid of micro features 510. In other embodiments, as is shown in FIG. 6, the micro features 510 can include through-holes that extend through the base 501 and segment 502 of the cover 500. The cover 500 can incorporate any of the micro features disclosed herein or combinations thereof.

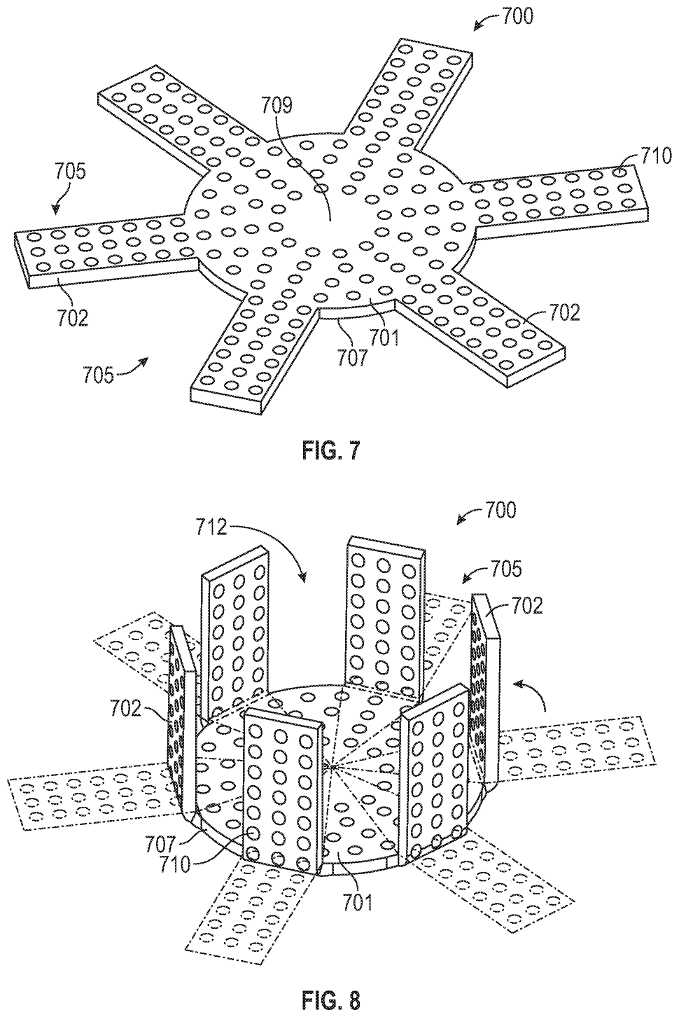

FIGS. 7 and 8 illustrate a foldable cover 700 in accordance with various embodiments. FIG. 7 shows the cover 700 in a flat state. FIG. 8 shows the cover 700 in a folded state. The cover 700 includes a base 701 having a generally circular shape. A multiplicity of segments 702 project outwardly from a perimeter 707 of the base 701. In the embodiment shown in FIGS. 7 and 8, each of the segments 702 has a constant width along the length of the segment 702. As shown, the cover 700 includes six segments 702. It is understood that the cover 700 can include more or fewer than six segments 702. When the segments 702 are displaced from a flat configuration to a folded configuration, as depicted in FIG. 8, a gap 705 is defined between adjacent segments 702.

The folded cover 700 has an opening 712 dimensioned to receive a distal end of a male-taper fitting of a luer device. As the male-taper fitting is advanced into the opening 712, some rotation occurs between the male-taper fitting and the cover 700. This rotation allows liquid disinfectant to be transferred from the micro features 710 on the segments 702 to portions of the side surface of the male-taper fitting that fall within the gap 705 between adjacent segments 702. In FIGS. 7 and 8, a central region 709 of the base 701 is devoid of micro features 710. In some embodiments, the central region 709 includes micro features 710. The cover 700 can incorporate any of the micro features disclosed herein or combinations thereof.

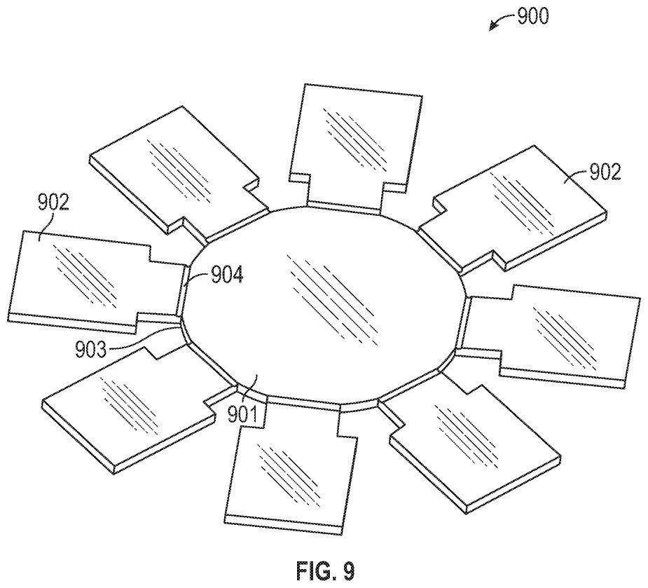

FIG. 9 illustrates a foldable cover 900 in accordance with various embodiments. The cover 900, shown in a flat state, includes a circular base 901 and a multiplicity of segments 902 projecting outwardly from a perimeter 903 of the base 901. A groove 904 is provided between the perimeter 903 of the base 901 and each of the segments 902. The groove 904, which can be a V-groove or channel, extends along the width of the portion of the segment 902 that connects with the perimeter 903 of the base 901. The groove 904 facilitates bending of the segments 902 relative to the base 901. For example, the groove 904 defines a hinge point of the cover 900, such that the cover 900 consistently bends in the same location when being inserted within the interior cavity of a disinfecting cap. According to some embodiments, the groove 904 can be part of a living hinge provided between the perimeter 903 of the base 901 and each of the segments 902.

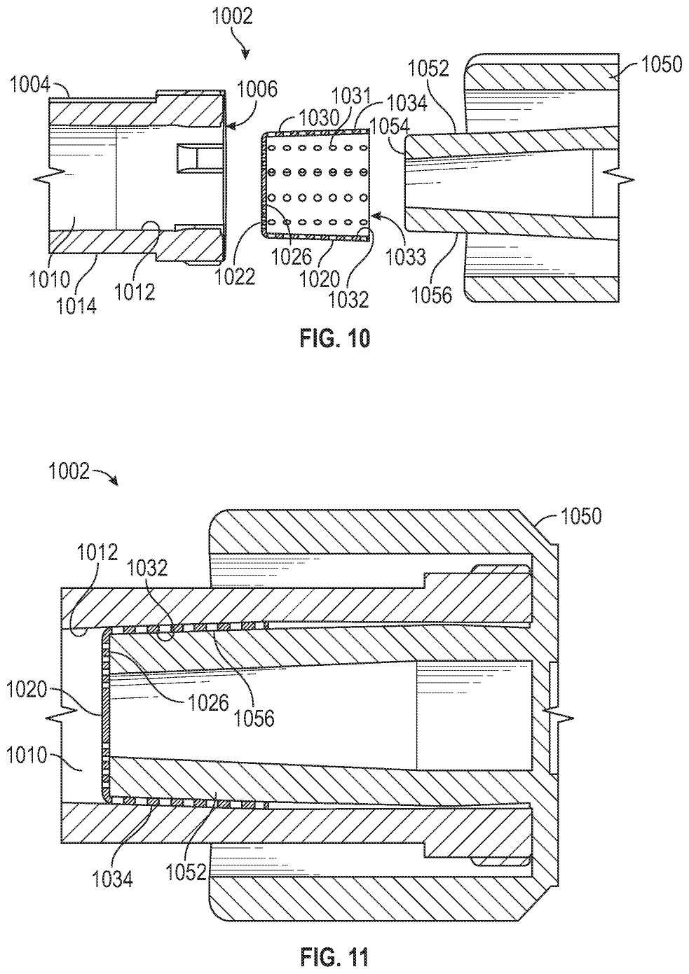

FIGS. 10 and 11 show a disinfectant system 1002 in accordance with various embodiments. The disinfectant system 1002 includes a cap 1004 having a construction similar to that shown in FIGS. 1-3. The portion of the cap 1004 shown in FIGS. 10 and 11 includes an opening 1006 to an interior cavity 1010, an inner surface 1012 defining the interior cavity 1010, and an outer surface 1014. The disinfectant system 1002 includes a cover 1020 configured as a basket comprising micro features 1031. According to some embodiments, the cover 1020 defines a molded strainer structure with micro features 1031 configured to retain a liquid disinfectant.

The micro features 1031 can be through-holes or other perforations extending through the sidewall 1030 of the cover 1020. The micro features 1031 can also be provided on the base 1022 of the cover 1020. In some embodiments, the micro features 1031, such as blind-holes, open channels or depressions, can be provided on the inner surface 1032 of the sidewall 1030 exclusively or in combination with such micro features 1031 provided on the inner surface 1026 of the base 1022.

When installed in the cap 1004, the cover 1020 is positioned near the opening 1006 as shown in FIG. 1, with an outer surface 1034 in contact with the inner surface 1012 of the cap 1004. As is best seen in FIG. 11, a clearance is added between the inner surface 1012 of the cap 1004 and the side surface 1056 of the male-taper fitting 1052 to accommodate the thickness of the sidewall 1030 of the cover 1020.

The cover 1020 includes an opening 1033 dimensioned to receive the distal end of a male-taper fitting 1052 of a luer device 1050. The inner surface 1032 of the sidewall 1030 of the cover 1020 preferably has a taper corresponding to a taper of the male-taper fitting 1052 (e.g., a 6% taper). As the cover 1020 is advanced over the distal end of the male-taper fitting 1052, liquid disinfectant retained by the micro features 1031 on the inner surface 1032 of the sidewall 1030 is transferred to the side surface 1056 of the male-taper fitting 1052. As the face 1054 of the male-taper fitting 1052 contacts the inner surface 1026 of the base 1022, liquid disinfectant retained by the micro features 1031 is transferred from the inner surface 1026 of the base 1022 to the face 1054 of the male-taper fitting 1052. Relative rotation between the inner surfaces 1032 and 1026 of the cover 1030 and the male-tapper fitting 1052 serves to wipe the liquid disinfectant across the side surface 1056 and face 1054 of the male-tapper fitting 1052.

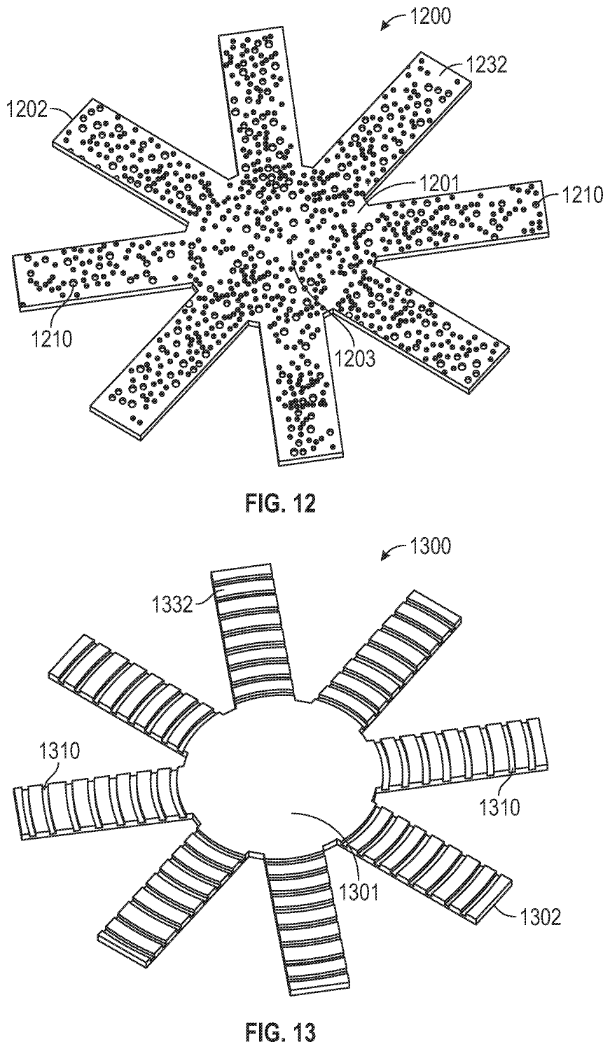

FIGS. 12-20 illustrate covers of a disinfectant system comprising various types of micro features in accordance with various embodiments. The covers shown in FIGS. 12-20 are presented in their flat state. In the embodiment shown in FIG. 12, the cover 1200 includes a base 1201 and a multiplicity of segments 1202 extending outwardly from a perimeter of the base 1201. An inner surface 1232 of the cover 1200 is shown in FIG. 12. The inner surface 1232 is the surface of the cover 1200 that contacts the male-taper fitting when the cover 1200 is positioned in its folded state within the inner cavity of a disinfectant cap. In the embodiment shown in FIG. 12, the inner surface 1232 of the cover 1200 comprises micro features 1210 in the form of pits and/or pores. As shown, the micro features 1210 are randomly arranged on the inner surface 1232 of the cover 1200. The pits and pores can be created by molding, etching, microreplicating, thermoforming, stamping, embossing, and many other processes. In some embodiments, the micro features 1210 distributed in the center region 1203 of the base 1201 can be smaller than the micro features 1210 on the segments 1202. The smaller micro features 1210 retain less liquid disinfectant than the larger micro features 1210, thereby further reducing or eliminating the risk of introducing liquid disinfectant into the lumen of the male-taper fitting.

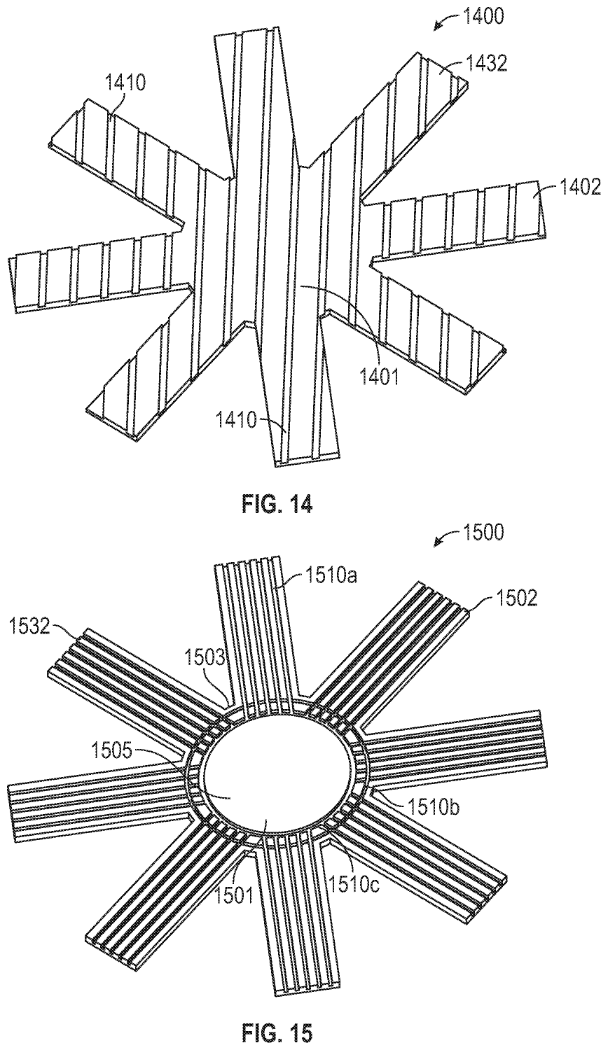

In the embodiment shown in FIG. 13, the cover 1300 includes a base 1301 and a multiplicity of segments 1302 projecting outwardly from a perimeter of the base 1301. An inner surface 1332 of the cover 1300 shown in FIG. 13 comprises micro features 1310 in the form of micro channels. More particularly, the micro features 1310 are concentric micro channels provided on each of the segments 1310. The micro channels can have dimensions in accordance with Equation 1 above. The micro channels can be created by molding, etching, microreplicating, thermoforming, stamping, embossing, and many other processes. In the embodiment shown in FIG. 13, the base 1301 does not incorporate the micro features 1310. In some embodiments, the base 1301 can incorporate the concentric micro channels 1310 or other micro features.

FIG. 14 shows an embodiment of a cover 1400 having an inner surface 1432 comprising micro features 1410 in the form of unidirectional micro channels. The unidirectional micro channels 1410 are oriented diagonally across the inner surface 1432 of the cover 1400. In the embodiment shown in FIG. 14, the micro channels 1410 extend across the base 1401 and the segments 1402 of the cover 1400. In some embodiments, the cover 1400 can include a stamped fluid transport film. The cross-sectional features of the micro channels can be implemented in accordance with Equation 1 above.

FIG. 15 shows an embodiment of a cover 1500 having an inner surface 1532 comprising micro features that define a network of interconnected micro channels. In the embodiment shown in FIG. 15, each of the segments 1502 incorporates a set of longitudinal micro channels 1510a. The longitudinal micro channels 1510a connect with a first concentric micro channel 1510b disposed near the perimeter 1503 of the base 1501. A second concentric micro channel 1510c is situated within the first concentric micro channel 1510b and connects with the longitudinal micro channels 1510a. The network of interconnected micro channels shown in the embodiment of FIG. 15 is configured to facilitate the transport of liquid disinfectant across the inner surface 1532 of the cover 1500. In the embodiment shown in FIG. 15, a center region 1505 of the base 1501 is devoid of micro features. In some embodiments, the network of interconnected micro channels or other micro features can be incorporated within the center region 1505 of the base 1501.

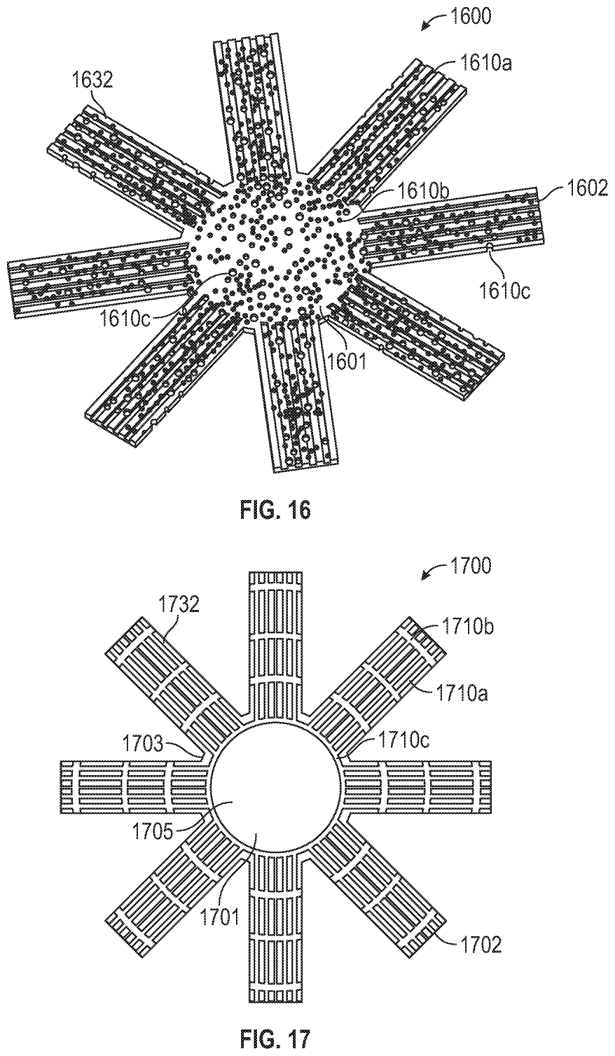

In the embodiment shown in FIG. 16, a cover 1600 includes an inner surface 1632 comprising a combination of different micro features. More particularly, each of the segments 1602 includes a combination of longitudinal micro channels 1610a and pits or pores 1610b. In addition to the micro channels 1610a and pits/pores 1610b, the segments 1602 can also include through-holes 1610c. The combination of micro features provided on the segments 1602 can be different from those provided on the base 1601. For example, the base 1601, which is devoid of the micro channels 1610a, can include one or both of pits/pores 1610b and through-hole 1610c.

FIG. 17 shows an embodiment of a cover 1700 having an inner surface 1732 comprising a combination of longitudinal micro channels 1710a and concentric micro channels 1710b which together form a network of interconnected micro channels. In the embodiment shown in FIG. 17, each of the segments 1702 includes a multiplicity of spaced-apart concentric micro channels 1710b that intersect with a multiplicity of longitudinal micro channels 1710a. A concentric micro channel 1710c is shown positioned near the perimeter 1703 of the base 1701. In the embodiment shown in FIG. 17, a center region 1705 of the base 1701 is devoid of micro features. In some embodiments, the network of interconnected micro channels or other micro features can be incorporated within the center region 1705 of the base 1701.

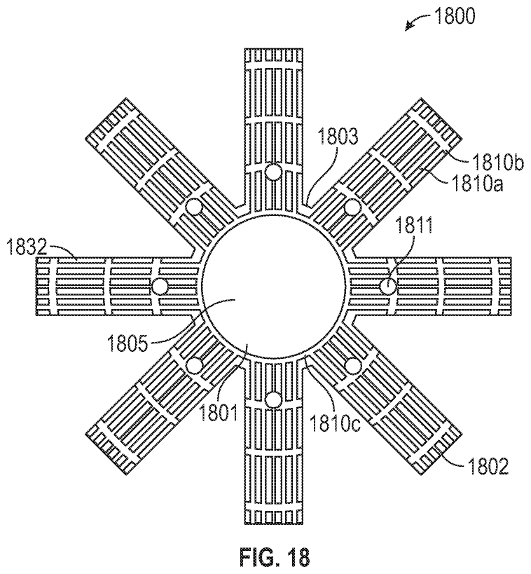

FIG. 18 shows an embodiment of a cover 1800 which is similar to that shown in FIG. 17. In the embodiment shown in FIG. 18, each of the segments 1802 include a multiplicity of spaced-apart concentric micro channels 1810b that intersect with a multiplicity of longitudinal micro channels 1810a. A concentric micro channel 1810c is positioned near the perimeter 1803 of the base 1801. Each of the segments 1802 is shown to include a through-hole 1811 situated near the perimeter 1803 of the base 1801. The through-holes 1811 are relatively large in size, and are not configured as micro features. The cover 1800 can be used in disinfectant systems that include a reservoir of liquid disinfectant within the cap. The through-holes 1811 allow a liquid disinfectant from the reservoir to pass from an outer surface (not shown) of the cover 1800 to the inner surface 1832. The network of interconnected micro channels is configured to distribute the liquid disinfectant passing through the through-holes 1811 across the inner surface 1832 of the segments 1802. In some embodiments, a central region 1805 of the base 1801 can incorporate a network of interconnected micro channels 1810a, 1810b or other micro features.

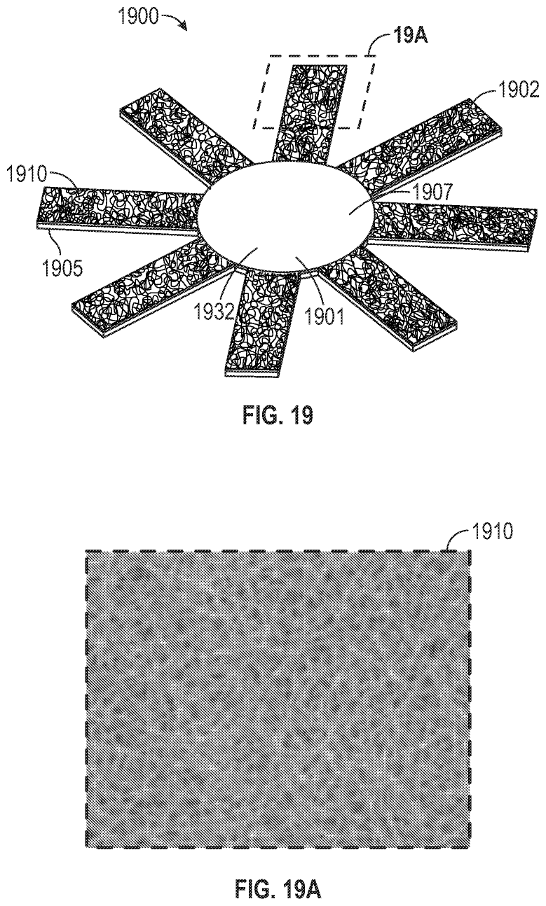

FIG. 19 shows an embodiment of a cover 1900 comprising a substrate 1905 and a layer of material 1910 affixed to an inner surface 1932 of the cover 1900. The layer of material 1910 comprises micro features. In particular, the layer of material 1910 shown in FIG. 19A includes a porous fiber-like material, such as a nonwoven material. The substrate 1905 is typically formed from plastic, but may alternatively be formed from metal. The layer of material 1910 comprising the micro features can be affixed to the substrate 1905 using an attachment method, such as adhesive (hot melt, pressure sensitive adhesive, epoxy, etc.), energy welding (hot plate, ultrasonics, spin welding) or mechanical attachment (crimping, engagement features, etc.). In some embodiments, the layer of material 1910 comprising micro features is affixed to the inner surface (not shown) of the segments 1902, as illustrated in FIG. 19. In other embodiments, the layer of material 1910 comprising micro features is affixed to the inner surface of the segments 1902 and the inner surface 1907 of the base 1901. According to other embodiments, the entire cover 1900 is formed from a porous fiber-like material, such as a nonwoven material.

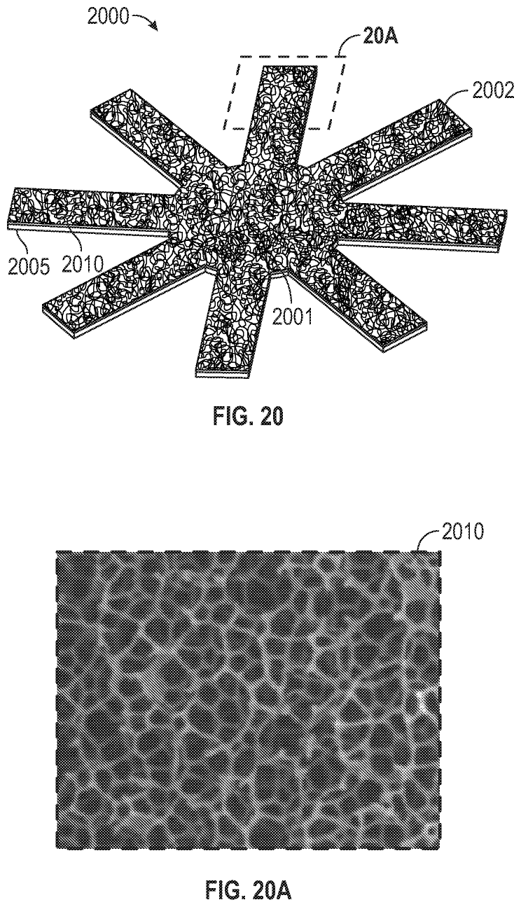

FIG. 20 shows an embodiment of a cover 2000 comprising a substrate 2005 and a layer of material 2010 comprising micro features affixed to an inner surface (not shown) of the cover 2000. The layer of material 2010 shown in FIG. 20A comprises a sponge material, which may be natural or synthetic sponge material. The substrate 2005 is typically formed from plastic, but may alternatively be formed from metal. The layer of material 2010 comprising the micro features can be affixed to the substrate 2005 using an attachment method, such as adhesive (hot melt, pressure sensitive adhesive, epoxy, etc.), energy welding (hot plate, ultrasonics, spin welding) or mechanical attachment (crimping, engagement features, etc.). In some embodiments, the layer of material 2010 comprising micro features is affixed to the inner surface of the segments 2002. In other embodiments, the layer of material 2010 comprising micro features is affixed to the inner surface of the segments 2002 and the inner surface of the base 2001, as illustrated in FIG. 20. According to other embodiments, the entire cover 2000 is formed from a sponge material.

FIGS. 21-26 illustrate an apparatus and method for making a disinfecting system in accordance with various embodiments. FIGS. 21-26 illustrate an apparatus and method of automatically forming a folded cover from an initially flat cover during a process of inserting the cover into an interior cavity of a disinfecting cap. The method of making a disinfecting system as illustrated in FIGS. 21-26 can be an automated process, a manual process or a combined automated/manual process.

As is shown in FIG. 21, a cover 2100 in a flat state is positioned relative to an insertion tool 2110. The cover 2100 can be any of the foldable covers previously described hereinabove. The cover 2100 includes a base 2101 and a multiplicity of segments 2102 projecting outwardly from a perimeter of the base 2101. The cover includes micro features 2121 which, in this representative example, are shown as through-holes.

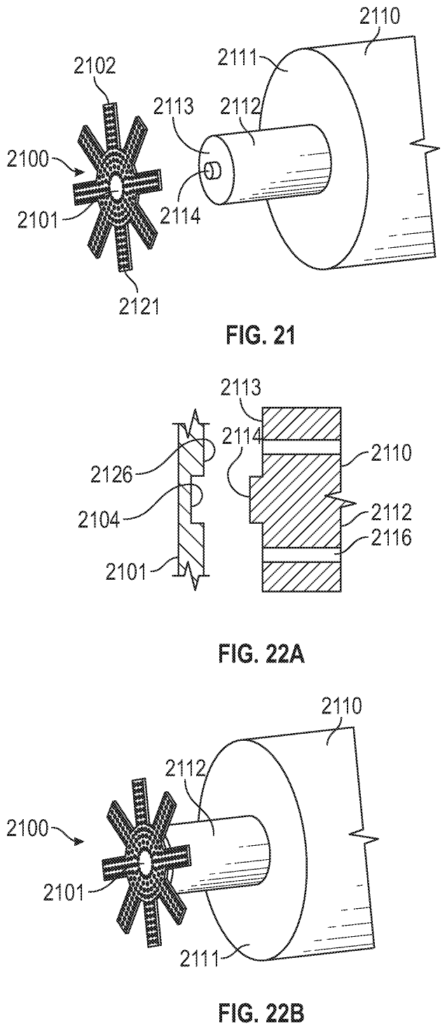

The insertion tool 2110 includes a shaft 2112 having an end surface 2113 configured to engage the base 2101 of the cover 2100. As is shown in FIG. 22A, the end surface 2113 of the shaft 2112 includes a pillar 2114. The base 2101 of the cover 2100 includes a recess 2104 configured to receive the pillar 2114. With the pillar 2114 inserted within the recess 2104, an inner surface 2126 of the base 2101 is held against the end surface 2113 of the shaft 2112. In some embodiments, a vacuum can be created at the end surface 2113 using channels 2116 to firmly hold the cover 2100 against the end surface 2113 of the shaft 2112. In other embodiments, an electrostatic force can be created between the shaft 2112 and the base 2101 of the cover 2100 to firmly hold the cover 2100 against the end surface 2113 of the shaft 2112.

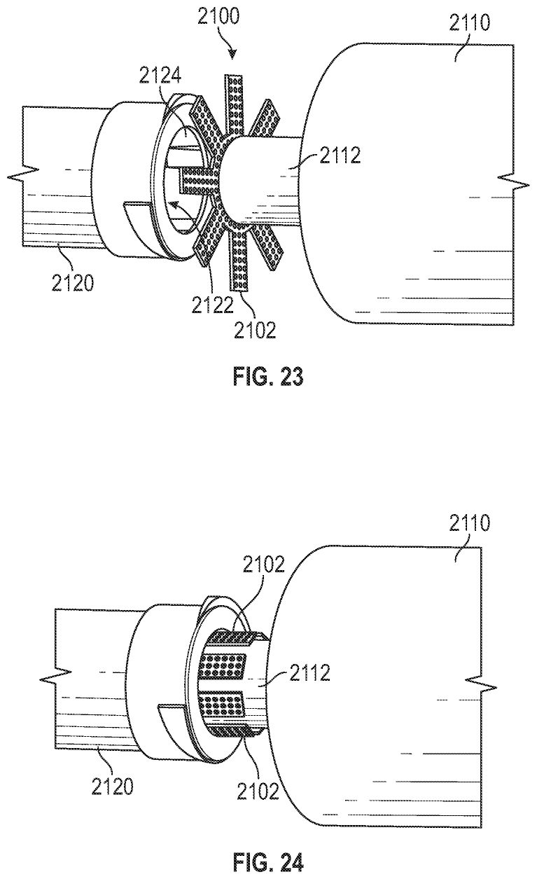

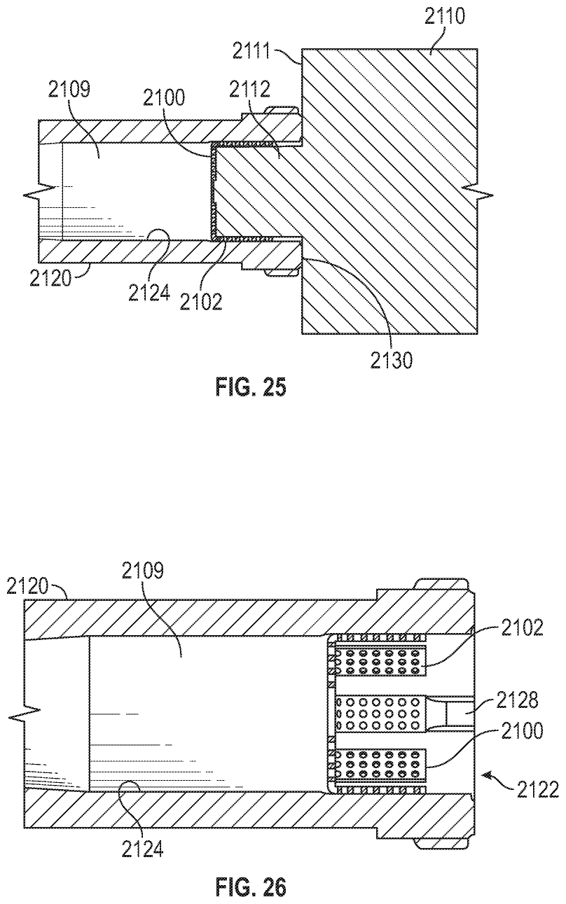

In FIG. 23, the cover 2100 held firmly by the insertion tool 2110 is positioned relative to an opening 2122 of a disinfectant cap 2120. The shaft 2112 of the insertion tool 2110 has a size that allows it to pass through the opening 2122 of the disinfectant cap 2120 and into an interior cavity 2109 (see FIGS. 25-26) of the cap 2120. For example, the shaft 2112 can have a cylindrical shape with a diameter that is less than a diameter of the interior cavity 2109 of the cap 2120. As is shown in FIG. 24, the flat cover 2100 shown in FIG. 23 is forced through the opening 2122 and into the interior cavity 2109 of the cap 2120, causing the segments 2102 of the cover 2100 to automatically bend as the segments 2102 contact the inner surface 2124 of the cap 2120.

As is shown in FIG. 25, the folded cover 2100 is advanced into the interior cavity 2109 of the cap 2120 until a predetermined depth has been reached. The insertion depth of the folded cover 2100 can be determined by the length of the shaft 2112 relative to a lateral surface 2111 of the insertion tool 2110. Contact between the lateral surface 2111 and an end surface 2130 of the cap 2120 limits the insertion depth of the folded cover 2100 within the interior cavity 2109 of the cap 2120. With the folded cover 2100 positioned at its predetermined depth within the interior cavity 2109, the shaft 2112 of the insertion tool 2110 is removed from the interior cavity 2109 of the cap 2120. As is shown in FIG. 26, a stop feature 2128 positioned near the opening 2122 of the cap 2120 prevents the folded cover 2100 from being pulled out of the interior cavity 2109 when the shaft 2112 of the insertion tool 2110 is removed from the interior cavity 2109 of the cap 2120.

### EXAMPLE EMBODIMENTS

The following example embodiments are provided, the numbering of which is not to be construed as designating levels of importance:

Embodiment A1 can include an article comprising:
a cap comprising an opening to an interior cavity, the cap having an inner surface defining the interior cavity and an outer surface;
a cover disposed within the interior cavity of the cap near the opening, the cover comprising:
   a base having an inner surface facing the cap opening and an opposite outer surface;
   a sidewall extending from a perimeter of the base toward the cap opening, the sidewall having an outer surface facing the inner surface of the cap and an opposite inner surface; and
   micro features on at least the inner surface of the sidewall;
wherein the cover is displaceable into the interior cavity of the cap.

In Embodiment A2, the article of Embodiment A1 can include, wherein the inner surfaces of the base and sidewall are dimensioned to cover at least a portion of a distal end of a male-taper fitting of a luer device.

In Embodiment A3, the article of at least one of Embodiments A1 and A2 can include, wherein the micro features are on the inner surface of the sidewall and the inner surface of the base.

In Embodiment A4, the article of at least one of Embodiments A1-A3 can include, wherein the base comprises a plurality of notches spaced apart from each other along the perimeter of the base.

In Embodiment A5, the article of at least one of Embodiments A1-A4 can include, wherein the sidewall of the cover is in direct contact with the inner surface of the cap.

In Embodiment A6, the article of at least one of Embodiments A1-A5 can include, wherein the inner surface of the cap tapers inward from the cap opening.

In Embodiment A7, the article of at least one of Embodiments A1-A6 can include, comprising one or more stop features near the opening of the cap.

In Embodiment A8, the article of at least one of Embodiments A1-A7 can include, wherein the sidewall of the cover extends completely around the perimeter of the base.

In Embodiment A9, the article of at least one of Embodiments A1-A8 can include, wherein the cover comprises a perforated basket.

In Embodiment A10, the article of at least one of Embodiments A1-A7 can include, wherein the sidewall of the cover comprises a plurality of segments that extend from the perimeter of the base towards the cap opening.

In Embodiment A11, the article of Embodiment A10 can include, wherein the sidewall comprises six segments symmetrically arranged around the perimeter of the base.

In Embodiment A12, the article of Embodiment A10 can include, wherein the sidewall comprises eight segments symmetrically arranged around the perimeter of the base.

In Embodiment A13, the article of at least one of Embodiments A10-A12 can include, wherein the segments are configured to exert a force against the inner surface of the cap.

In Embodiment A14, the article of at least one of Embodiments A10-A13 can include, wherein adjacent segments contact each other.

In Embodiment A15, the article of at least one of Embodiments A10-A14 can include, wherein a gap is defined between adjacent segments.

In Embodiment A16, the article of at least one of Embodiments A10-A15 can comprising a groove at a hinge point between the base and each of the segments.

In Embodiment A17, the article of at least one of Embodiments A1-A16 can include, wherein the sidewall of the cover is configured to bend at the base when the cover is disposed within the interior cavity of the cap.

In Embodiment A18, the article of at least one of Embodiments A1-A17 can include, wherein the micro features comprise at least one of blind-holes, open channels, depressions, or combinations thereof.

In Embodiment A19, the article of at least one of Embodiments A1-A17 can include, wherein the micro features comprise at least one of through-holes, slots, apertures, perforations, or combinations thereof.

In Embodiment A20, the article of at least one of Embodiments A1-A17 can include, wherein the micro features comprise at least one of blind-holes, open channels, depressions, through-holes, slots, apertures, perforations, or combinations thereof.

In Embodiment A21, the article of at least one of Embodiments A1-A20 can include, wherein the micro features are of a uniform size and shape.

In Embodiment A22, the article of at least one of Embodiments A1-A21 can include, wherein the cover comprises at least one of a fibrous material and sponge having micro features throughout.

In Embodiment A23, the article of at least one of Embodiments A1-A22 can include, wherein the cover comprises a layer of material affixed to at least the inner surface of the sidewall, the layer of material comprising the micro features.

In Embodiment A24, the article of at least one of Embodiments A1-A23 can include, wherein the micro features are arranged in a pattern.

In Embodiment A25, the article of at least one of Embodiments A1-A23 can include, wherein the micro features are randomly arranged.

In Embodiment A26, the article of at least one of Embodiments A1-A25 can further comprise a cleaning agent in at least a portion of the micro features.

In Embodiment A27, the article of at least one of Embodiments A1-A26 can further comprise cleaning agent in the interior cavity of the cap.

Embodiment B1 can include a disinfectant system comprising:
a cap comprising an opening to an interior cavity, the cap having an inner surface defining the interior cavity and an outer surface;
a cover disposed within the interior cavity of the cap near the opening, the cover comprising:
   a base having an inner surface facing the cap opening and an opposite outer surface;
   a sidewall extending from a perimeter of the base toward the cap opening, the sidewall having an outer surface facing the inner surface of the cap and an opposite inner surface;
   micro features on the inner surfaces of the base and the sidewall; and
   disinfectant in the interior cavity of the cap;
wherein the cover is displaceable into the interior cavity of the cap.

In Embodiment B2, the Embodiment B1 can include, wherein the cap comprises a closed end opposite the opening, and the disinfectant is held in the interior cavity between the base of the cover and the closed end of the cap.

In Embodiment B3, the system of at least one of B1 and B2 can include, wherein the disinfectant flows into the cover when the cover is displaced into the interior cavity of the cap.

In Embodiment B4, the system of at least one of B1-B3 can include, wherein disinfectant is held within the micro features.

Embodiment C1 can include a method of making the article of Embodiment A1 comprising:
providing a foldable cover that comprises the base and sidewall of the cover in a single plane;
placing the foldable cover over the opening of the cap such that the base is centered on the opening; and
pushing the foldable cover through the cap opening, the sidewall bending at the perimeter of the base as the foldable cover progresses into the interior cavity.

In Embodiment C2, the method of Embodiment C1 can comprise:
holding the base of the foldable cover against a shaft of an insertion tool; and
forcing the foldable cover and the shaft through the cap opening and into the interior cavity of the cap.

In Embodiment C3, the method of Embodiment C2 can comprise:
forcing the foldable cover to a predefined depth within the interior cavity;
releasing the shaft from the foldable cover after reaching the predefined depth; and
removing the shaft from the interior cavity of the cap.

In Embodiment C4, the method of at least one of Embodiments C2 and C3 can include, wherein the foldable cover is held against the shaft via at least one of a vacuum and an electrostatic force.

In Embodiment C5, the method of at least one of Embodiments C2-C4 can include, wherein the shaft comprises a pillar and the base of the foldable cover comprises a recess dimensioned to receive the pillar.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g. 1 to 5 includes 1, 1.5, 2, 2.75, 3, 3.80, 4, and 5) and any range within that range.

Various modifications and alterations of these embodiments will be apparent to those skilled in the art and it should be understood that this scope of this disclosure is not limited to the illustrative embodiments set forth herein. For example, the reader should assume that features of one disclosed embodiment can also be applied to all other disclosed embodiments unless otherwise indicated.

1. An article, comprising:
a cap comprising an opening to an interior cavity, the cap having an inner surface defining the interior cavity and an outer surface;
a cover disposed within the interior cavity of the cap near the opening, the cover comprising:
a base having an inner surface facing the cap opening and an opposite outer surface;
a sidewall extending from a perimeter of the base toward the cap opening, the sidewall having an outer surface facing the inner surface of the cap and an opposite inner surface; and
micro features on at least the inner surface of the sidewall;
wherein the cover is displaceable into the interior cavity of the cap.

2. (canceled)

3. The article of claim 1, wherein the micro features are on the inner surface of the sidewall and the inner surface of the base.

4. (canceled)

5. The article of claim 1, wherein the sidewall of the cover is in direct contact with the inner surface of the cap.

6. (canceled)

7. The article of claim 1, comprising one or more stop features near the opening of the cap.

8. The article of claim 1, wherein the sidewall of the cover extends completely around the perimeter of the base.

9. The article of claim 1, wherein the cover comprises a perforated basket.

10. The article of claim 1, wherein the sidewall of the cover comprises a plurality of segments that extend from the perimeter of the base towards the cap opening.

11. (canceled)

12. (Canceled)

13. The article of claim 10, wherein the segments are configured to exert a force against the inner surface of the cap.

14. The article of claim 10, wherein adjacent segments contact each other.

15. The article of claim 10, wherein a gap is defined between adjacent segments.

16. The article of claim 10, comprising a groove at a hinge point between the base and each of the segments.

17. The article of claim 1, wherein the sidewall of the cover is configured to bend at the base when the cover is disposed within the interior cavity of the cap.

18. (canceled)

19. (canceled)

20. The article of claim 1, wherein the micro features comprise at least one of blind-holes, open channels, depressions, through-holes, slots, apertures, perforations, or combinations thereof.

21. (canceled)

22. (canceled)

23. The article of claim 1, wherein the cover comprises a layer of material affixed to at least the inner surface of the sidewall, the layer of material comprising the micro features.

24. (canceled)

25. (canceled)

26. The article of claim 1, further comprising a cleaning agent in at least a portion of the micro features.

27. (canceled)

28. A disinfectant system, comprising:
a cap comprising an opening to an interior cavity, the cap having an inner surface defining the interior cavity and an outer surface;
a cover disposed within the interior cavity of the cap near the opening, the cover comprising:
a base having an inner surface facing the cap opening and an opposite outer surface;
a sidewall extending from a perimeter of the base toward the cap opening, the sidewall having an outer surface facing the inner surface of the cap and an opposite inner surface;
micro features on the inner surfaces of the base and the sidewall; and
disinfectant in the interior cavity of the cap;
wherein the cover is displaceable into the interior cavity of the cap.

29. The system of claim 28, wherein the cap comprises a closed end opposite the opening, and the disinfectant is held in the interior cavity between the base of the cover and the closed end of the cap.

30. (canceled)

31. The system of claim 28, wherein disinfectant is held within the micro features.

32. A method of making the article of claim 1, comprising:
providing a foldable cover that comprises the base and sidewall of the cover in a single plane;
placing the foldable cover over the opening of the cap such that the base is centered on the opening; and
pushing the foldable cover through the cap opening, the sidewall bending at the perimeter of the base as the foldable cover progresses into the interior cavity.

33. The method of claim 32, comprising:
holding the base of the foldable cover against a shaft of an insertion tool; and
forcing the foldable cover and the shaft through the cap opening and into the interior cavity of the cap.

34. The method of claim 33, comprising:
forcing the foldable cover to a predefined depth within the interior cavity;
releasing the shaft from the foldable cover after reaching the predefined depth; and
removing the shaft from the interior cavity of the cap.

35. (canceled)

36. (canceled)
